(19) United States
(12) Patent Application Publication
(10) Pub. No.: US 2014/0135513 A1
(43) Pub. Date: May 15, 2014

(54) EO REACTOR, PROCESS AND THERMOCOUPLE PLACEMENT

(71) Applicant: SHELL OIL COMPANY, Houston, TX (US)

(72) Inventor: Paul Michael MCALLISTER, Houston, TX (US)

(73) Assignee: SHELL OIL COMPANY, Houston, TX (US)

(21) Appl. No.: 13/678,822

(22) Filed: Nov. 16, 2012

Related U.S. Application Data
(60) Provisional application No. 61/726,875, filed on Nov. 15, 2012.

(51) Int. Cl. C07D 301/08

(52) U.S. Cl. CPC C07D 301/08
USPC 549/534; 549/523; 568/867; 564/477

(57) ABSTRACT

Techniques are provided for determining the proper way to load thermocouple reactor tubes in multi-tubular ethylene oxide reactors containing a large number of reactor tubes containing silver catalysts. In these techniques, it is necessary to adjust the pressure drop so that oxygen conversion by thermocouple reactor tubes will closely match that of non-thermocouple reactor tubes.

### PRIORITY CLAIM

The present application claims priority from U.S. provisional application 61/726,875, filed 15 Nov. 2012, which is incorporated herein by reference.

### FIELD OF INVENTION

The invention relates to fixed-bed, multi-tubular reactor systems for the manufacture of ethylene oxide where certain of the tubes contain a thermocouple. The invention also relates to the use of the reactor systems in the manufacture of ethylene oxide, and chemicals derivable from ethylene oxide.

### BACKGROUND OF THE INVENTION

Ethylene oxide is an important industrial chemical used as a feedstock for making such chemicals as ethylene glycol, ethylene glycol ethers, ethanol amines and detergents. One method for manufacturing ethylene oxide is by epoxidation of ethylene—i.e., the catalyzed partial oxidation of ethylene with oxygen yielding ethylene oxide. The ethylene oxide so manufactured may be reacted with water, an alcohol or an amine to produce ethylene glycol, ethylene glycol ether or an ethanol amine.

In ethylene epoxidation, a feedstream containing ethylene and oxygen is passed over a bed of catalyst contained within a reaction zone that is maintained at certain reaction conditions. The relatively large heat of reaction makes adiabatic operation at reasonable operation rates impossible. While some of the generated heat may leave the reaction zone as sensible heat, most of the heat needs to be removed through the use of a coolant. The temperature of the catalyst needs to be controlled carefully as the relative rates of epoxidation and combustion to carbon dioxide and water are highly temperature dependent. The temperature dependency together with the relatively large heat of reaction can easily lead to run-away reactions.

A commercial ethylene epoxidation reactor is generally in the form of a shell-and-tube heat exchanger, in which a plurality of substantially parallel elongated, relatively narrow tubes are filled with catalyst particles to form a packed bed, and in which the shell contains a coolant. Irrespective of the type of epoxidation catalyst used, in commercial operation the internal tube diameter is frequently in the range of from 20 to 60 mm, and the number of tubes per reactor may range in the thousands, for example up to 12,000. Reference is made to U.S. Pat. No. 4,921,681 and U.S. Pat. App. No. 2009/0234144.

With the catalyst bed present in narrow tubes, axial temperature gradients over the catalyst bed and hot spots are practically eliminated. In this way, careful control of the temperature of the catalyst can be achieved and conditions leading to run-away reactions are substantially avoided. The temperature in the reactor tubes is often measured by the use of thermocouples placed in a few of the many thousand reactor tubes. It is extremely important to know the actual temperatures within the reactor tubes so that all the components and rates may be controlled to achieve the desired selectivity and productivity. Therefore, it is vitally important that the temperature within the reactor tubes be measured accurately and that such measurement reflects the temperature in all of the reactor tubes, not just the reactor tubes containing thermocouples.

### SUMMARY OF THE INVENTION

Multi-tubular reactors are typically used for production of ethylene oxide and other petrochemicals that generate heat due to reaction. As a means of monitoring the performance of these reactors, axially positioned thermocouples are placed in selected reactor tubes within the reactor. As used herein, the term “thermocouple reactor tube” will refer to a reactor tube comprising a thermocouple. Additionally, as used herein, the term “non-thermocouple reactor tube” will refer to a reactor tube that does not comprise a thermocouple. Typically, one reactor may have from 5 up to 50 thermocouple reactor tubes out of 1,000-12,000 total reactor tubes. For ethylene oxide reactors, the typical reactor tube inner diameter ranges from 30 to 55 millimeters (“mm”) and the thermocouple outer diameter usually ranges from 3 to 6 mm. The thermocouples run the entire length of the thermocouple reactor tubes and are centered within the thermocouple reactor tubes by positioning devices. Each thermocouple typically has 5-10 measurement points along its length to allow the operator to observe the temperature profile in the catalyst bed. These data assist the operator in starting up the reactor smoothly, monitoring for runaway reaction, or hotspots, and quickly observing upsets in operation.

In order for thermocouple reactor tubes to provide useful data and to prevent them from causing problems such as runaways and post ignitions, it is important to properly load the thermocouple reactor tubes and adjust the pressure drop across them so that the conversion of reactants across the thermocouple reactor tubes is very close to that of the non-thermocouple reactor tubes.

In prior procedures for loading such thermocouple reactor tubes, it was simply specified that the pressure drop of each thermocouple reactor tube be adjusted to 103-108% of the average of the non-thermocouple reactor tubes in the reactor after applying a velocity correction factor.

Measuring the pressure drop is normally done with a fixed steady flow. Because the area in a thermocouple reactor tube is smaller due to the space of the thermocouple, the measurement of thermocouple reactor tubes must be corrected to the non-thermocouple reactor tubes by the velocity correction factor.

Velocity Correction Factor=[(A²−B²)/A²]^{1.83}

### Where

A=Inner Diameter (“ID”) of thermocouple reactor tube in mm (inches)

B=Outer Diameter (“OD”) of thermocouple in mm (inches)

Thus, the required pressure drop of the thermocouple reactor tubes was obtained as follows:
define the highest pressure drop in the non-thermocouple reactor tubes;
calculate the velocity correction factor; and
divide the highest pressure drop in the non-thermocouple reactor tubes by the velocity correction factor.

The major problem with this guidance was that it did not require that an adequate amount of active catalyst be loaded into the thermocouple reactor tubes and the flow necessarily adjusted such that the conversion of reactants would be equivalent between the thermocouple reactor tubes and non-thermocouple reactor tubes. This is corrected by the present invention as further defined below.

Accordingly, a technique is described herein for determining the proper way to load thermocouple reactor tubes and to adjust the pressure drop so that oxygen conversion by thermocouple reactor tubes will closely match that of non-thermocouple reactor tubes. The important point is that the presence of the thermocouple in the thermocouple reactor tube influences both the amount of active catalyst that can be loaded in the thermocouple reactor tube and the resistance to flow of gas through the thermocouple reactor tube. This invention provides an improved method for specifying the target pressure drop for thermocouple reactor tubes and provides an algorithm for insuring that thermocouple reactor tubes are properly loaded in commercial reactors.

In one embodiment, the present invention relates to a process for improving the control of a tubular reactor for the preparation of ethylene oxide wherein a gaseous stream comprising ethylene and an oxygen-containing gas is passed through a fixed bed multi-tubular reactor which comprises: (a) thermocouple reactor tubes containing catalyst and an inert material loaded on top of the catalyst and (b) non-thermocouple reactor tubes containing catalyst, wherein prior to start-up and loading of the reactor tubes:
a. the gas flow per unit mass of catalyst in the thermocouple reactor tubes is specified as being substantially equal to the gas flow per unit mass of catalyst in the non-thermocouple reactor tubes;
b. the expected pressure drop and loading density of the catalyst is calculated to determine the expected differential in gas flow and/or pressure drop between the thermocouple reactor tubes and the non-thermocouple reactor tubes;
c. the pressure drop characteristics of the inert material is established to determine the amount of inert material to be loaded on top of the catalyst in the thermocouple reactor tubes and in the non-thermocouple reactor tubes to achieve the equivalent gas flow per unit mass of catalyst in the non-thermocouple reactor tubes and in the thermocouple reactor tubes under normal operating conditions; and
d. a target pressure drop value across the thermocouple reactor tubes is calculated that should be achieved in pressure drop checks that are to be conducted on the reactor tubes after loading is completed.

Following this calculation, the reactor tubes are then loaded with the catalyst and an inert material as determined by the prior calculations, and the pressure drop across the thermocouple reactor tubes and the non-thermocouple reactor tubes is measured to determine any difference in pressure drop across the two types of reactor tubes. Then the amount of inert material in the thermocouple reactor tubes is adjusted to achieve the desired pressure drop target relative to that found in the non-thermocouple reactor tubes.

In addition, the invention also provides a method of preparing ethylene glycol, an ethylene glycol ether or an ethanol amine comprising obtaining ethylene oxide by the process for the epoxidation of ethylene according to this invention, and converting the ethylene oxide into ethylene glycol, the ethylene glycol ether, or the ethanol amine.

### DESCRIPTION OF THE DRAWINGS

FIG. 1 is a cross-section of a non-thermocouple reactor tube filled with catalyst.

FIG. 2 is a cross-section of a thermocouple reactor tube containing catalyst and an inert material.

FIG. 3 is a schematic representation of an ethylene oxide manufacturing process which includes certain novel aspects of the invention.

FIG. 4 is a plot of Time of Operation versus Outlet Oxygen Content per Example 1.

FIG. 5 is a plot of Flow per unit Mass versus Oxygen Concentration per Example 1.

### DETAILED DESCRIPTION OF THE INVENTION

Epoxidation catalysts which comprise silver in quantities between 100 and 500 g/kg catalyst and additionally a promoter component selected from rhenium, tungsten, molybdenum and chromium have been used commercially for many years. Particularly advantageous is the use of such epoxidation catalysts having silver in quantities of at least 150 g/kg catalyst.

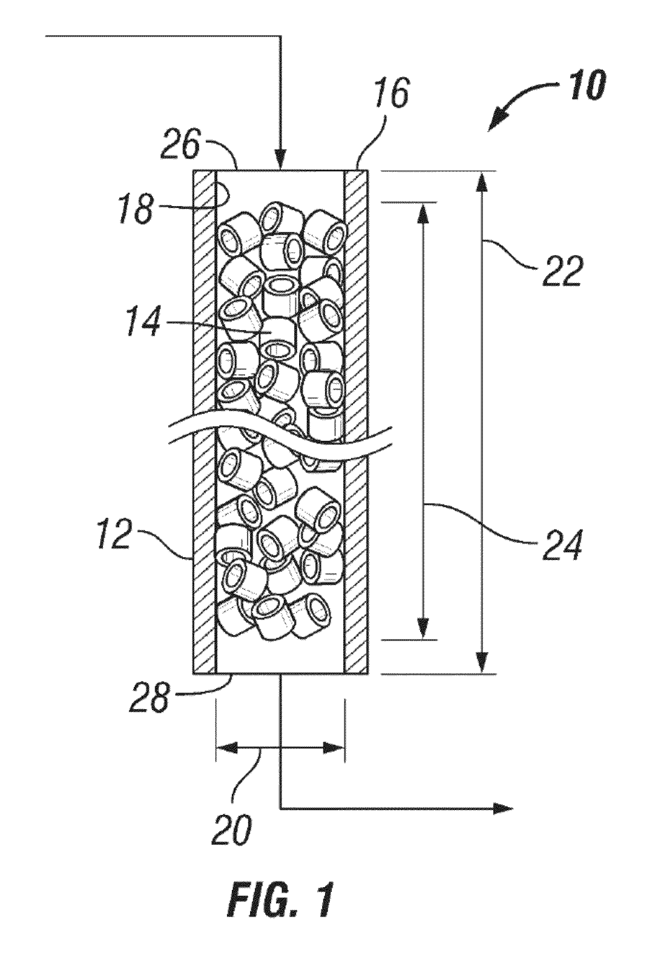

Referring to FIG. 1, a non-thermocouple reactor tube 10 is depicted comprising elongated tube 12. Elongated tube 12 has a tube wall 16 with an inside tube surface 18 and internal tube diameter 20 that define a reaction zone, wherein is contained catalyst bed 14. Elongated tube 12 has a tube length 22 and the catalyst bed 14 contained within the reaction zone has a bed depth 24.

The internal tube diameter 20 is typically above 30 mm, preferably between 30 and 55 mm, and at most 80 mm. Preferably, the tube length 22 is at least 3 meters (“m”), more preferably at least 5 m. Preferably the tube length 22 is at most 25 m, more preferably at most 20 m. Preferably, the wall thickness of the elongated tube 12 is at least 0.5 mm, more preferably at least 0.8 mm, and in particular at least 1 mm. Preferably, the wall thickness of the elongated tube 12 is at most 10 mm, more preferably at most 8 mm, and in particular at most 5 mm.

Outside the bed depth 24, the elongated tube 12 may contain a separate bed of particles of a non-catalytic or inert material (not depicted) for the purpose of, for example, heat exchange with a feedstream and/or another such separate bed for the purpose of, for example, heat exchange with the reaction product. Similarly, outside the bed depth, the elongated tube 12 may contain a catalyst retention device (not depicted) to support the catalyst. Preferably, the bed depth 24 is at least 3 m, more preferably at least 5 m. Preferably, the bed depth 24 is at most 25 m, more preferably at most 20 m. The elongated tube 12 further has an inlet tube end 26 into which a feedstream comprising ethylene and oxygen can be introduced and an outlet tube end 28 from which a reaction product comprising ethylene oxide and ethylene can be withdrawn. It is noted that the ethylene in the reaction product, if any, is ethylene of the feedstream which passes through the reactor zone unconverted. Typical conversions of the ethylene exceed 10 mole percent, but, in some instances, the conversion may be less.

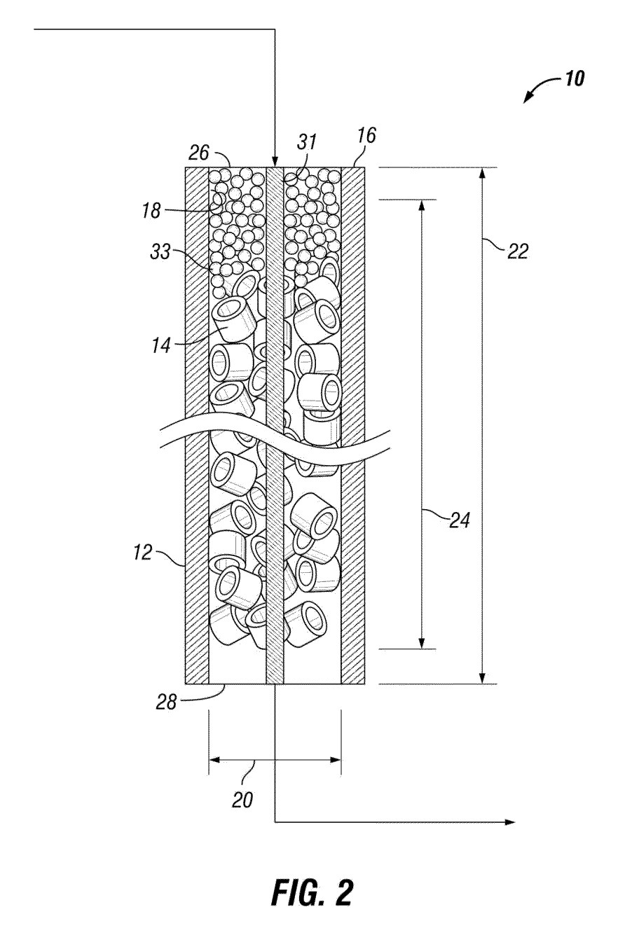

Reference is now made to FIG. 2, which depicts a thermocouple reactor tube 30 comprising a thermocouple 31 and a reactor tube having those additional features previously described herein with respect to non-thermocouple reactor tubes and depicted in FIG. 1. The thermocouple 31 will enter the reactor head through a nozzle and be of sufficient length to extend from the inlet end of the thermocouple reactor tube to near the outlet or catalyst retention device of the same tube. Each thermocouple will include multiple temperature points to allow determination of temperatures at several points in the catalyst bed along the length of the reactor tube. The catalyst retention device may also be used to keep the thermocouple in the proper position. A typical thermocouple will have 5 to 10 temperature points. The common method of installing and securing a thermocouple is to insert it through a threaded compression fitting that is welded to a flange installed on the reactor head. Within the head, the thermocouples are secured to prevent damage due to excessive vibration. Outside the reactor, the end of the thermocouple assembly extends into a weatherproof junction box, where connection is made to the plant control system so that the catalyst temperatures can be monitored. Numerous types of thermocouple materials exist and should be selected for the desired application. Chromel/Alumel thermocouples are suitable for the measurement of temperatures in ethylene oxide reactors and the material for the thermowells should be selected so as to be resistance to corrosion in the process gas stream. A typical reactor will contain a total of about 1,000 to about 12,000 reactor tubes, of which reactor tubes between 5 and 50, preferably between 5 and 30 will contain thermocouples. As for the selection of the specific reactor tubes that will contain a thermocouple, the location of thermocouples for the reactor tubes should be selected to minimize differences in mass flow and heat flux between the thermocouple reactor tubes and non-thermocouple reactor tubes in order to permit meaningful and representative measurements. Also noted as 33 is the inert material that is included to attain the desired pressure drop. The key aspect of the present invention is to prepare the thermocouple reactor tubes in such a way that they accurately measure the temperature that is found in all of the reactor tubes, regardless of whether they have a thermocouple or not. One such illustration to assure that is the case is shown in Example 1 below.

As mentioned above, both thermocouple and non-thermocouple reactor tubes contain a catalyst bed comprising catalyst particles. The catalyst particles comprise silver and a promoter component deposited on a carrier. In the normal practice of this invention, a major portion of the catalyst bed comprises the catalyst particles. By “a major portion” it is meant that the ratio of the weight of the catalyst particles to the weight of all the particles contained in the catalyst bed, is at least 0.50, in particular at least 0.8, but preferably at least 0.85 and, most preferably at least 0.9. Particles which may be contained in the catalyst bed other than the catalyst particles are, for example, inert particles or inert materials. Inert materials may also be used in the non-thermocouple reactor tubes in many cases near the inlet section of the tube to serve as a heatup zone. Temperatures in this zone are too low for significant reaction to occur, so some prefer not to spend money on catalyst for this section. In a few cases, inert materials are placed in a small zone near the outlet of the reactor tubes as well. This can help cool the gas before it exits the reactor tube or prevent catalyst particles from falling through the catalyst retention device positioned at the bottom of the reactor tube.

The carrier for use in this invention may be based on a wide range of materials. Such materials may be natural or artificial inorganic materials and they may include refractory materials, silicon carbide, clays, zeolites, charcoal and alkaline earth metal carbonates, for example calcium carbonate. Preferred are refractory materials, such as alumina, magnesia, zirconia and silica. The most preferred material is α-alumina. Typically, the carrier comprises at least 85% w, more typically at least 90% w, in particular at least 95%/w α-alumina, frequently up to 99.9%/w α-alumina, relative to the weight of the carrier. Other components of the α-alumina carrier may comprise, for example, silica, zirconium compounds such as zirconium oxide, alkali metal components, for example sodium and/or potassium components, and/or alkaline earth metal components, for example calcium and/or magnesium components. Binder materials are also normally included in carrier preparation.

The surface area of the carrier may suitably be at least 0.1 m²/g, preferably at least 0.3 m²/g, more preferably at least 0.5 m²/g, and in particular at least 0.6 m²/g, relative to the weight of the carrier; and the surface area may suitably be at most 10 m²/g, preferably at most 5 m²/g, and in particular at most 3 m²/g, relative to the weight of the carrier. “Surface area” as used herein is understood to relate to the surface area as determined by the B.E.T. (Brunauer, Emmett and Teller) method as described in Journal of the American Chemical Society 60 (1938) pp. 309-316. High surface area carriers, in particular when they are α-alumina carriers optionally comprising in addition silica, alkali metal and/or alkaline earth metal components, provide improved performance and stability of operation.

The water absorption of the carrier is typically in the range of from 0.2 to 0.8 g/g, preferably in the range of from 0.3 to 0.7 g/g. A higher water absorption may be in favor in view of a more efficient deposition of silver and further elements, if any, on the carrier by impregnation. However, at a higher water absorption, the carrier, or the catalyst made therefrom, may have lower crush strength. As used herein, water absorption is deemed to have been measured in accordance with ASTM C20, and water absorption is expressed as the weight of the water that can be absorbed into the pores of the carrier, relative to the weight of the carrier.

The carrier is typically a calcined, i.e. sintered, carrier, preferably in the form of formed bodies, the size of which is in general determined by the internal diameter of the elongated tube in which the catalyst particles are included in the catalyst bed. In general, the skilled person will be able to determine an appropriate size of the formed bodies. It is found very convenient to use formed bodies in the form of trapezoidal bodies, cylinders, saddles, spheres, doughnuts, and the like. The catalyst particles have preferably a generally hollow cylinder geometric configuration. The catalyst particles having a generally hollow cylinder geometric configuration 30 may have a length with a length of typically from 4 to 20 mm, more typically from 5 to 15 mm; an outside diameter typically from 4 to 20 mm, more typically from 5 to 15 mm; and inside diameter typically from 0.1 to 6 mm, preferably from 0.2 to 4 mm. Suitably the catalyst particles have a length and an inner diameter as described hereinbefore and an outside diameter of at least 7 mm, preferably at least 8 mm, more preferably at least 9 mm, and at most 20 mm, or at most 15 mm. The ratio of the length to the outside diameter is typically in the range of from 0.5 to 2, more typically from 0.8 to 1.2. While not wanting to be bound to any particular theory, it is believed, however, that the void space provided by the inside diameter of the hollow cylinder allows, when preparing the catalyst, for improved deposition of the catalytic component onto the carrier, for example by impregnation, and improved further handling, such as drying, and, when using the catalyst, it provides for a lower pressure drop over the catalyst bed. An advantage of applying a relatively small bore diameter is also that the shaped carrier material has higher crush strength relative to a carrier material having a larger bore diameter. Note that a variety of shapes may be employed and hollow cylinders are just one type of catalyst shape.

The preparation of the catalyst is known in the art and the known methods are applicable to the preparation of the catalyst particles which may be used in the practice of this invention. Methods of depositing silver on the carrier include impregnating the carrier with a silver compound containing cationic silver and performing a reduction to form metallic silver particles. Reference may be made, for example, to U.S. Pat. Nos. 5,380,697, 5,739,075, EP-A-266015, and U.S. Pat. No. 6,368,998, which US patents are incorporated herein by reference.

The reduction of cationic silver to metallic silver may be accomplished during a step in which the catalyst is dried, so that the reduction as such does not require a separate process step. This may be the case if the silver containing impregnation solution comprises a reducing agent, for example, an oxalate, a lactate or formaldehyde.

Appreciable catalytic activity is obtained by employing a silver content of the catalyst of at least 10 g/kg, relative to the weight of the catalyst. Preferably, the catalyst comprises silver in a quantity of from 50 to 500 g/kg, more preferably from 100 to 400 g/kg. In an embodiment, it is preferred to use catalysts having a high silver content. Preferably, the silver content of the catalyst may be at least 150 g/kg, more preferably at least 200 g/kg, and most preferably at least 250 g/kg, relative to the weight of the catalyst. Preferably, the silver content of the catalyst may be at most 500 g/kg, more preferably at most 450 g/kg, and most preferably at most 400 g/kg, relative to the weight of the catalyst. Preferably, the silver content of the catalyst is in the range of from 150 to 500 g/kg, more preferably from 200 to 400 g/kg, relative to the weight of the catalyst. For example, the catalyst may comprise silver in a quantity of 150 g/kg, or 180 g/kg, or 190 g/kg, or 200 g/kg, or 250 g/kg, or 350 g/kg, relative to the weight of the catalyst. In the preparation of a catalyst having a relatively high silver content, for example in the range of from 150 to 500 g/kg, on total catalyst, it may be advantageous to apply multiple depositions of silver.

The catalyst for use in this invention comprises a promoter component which comprises an element selected from rhenium, tungsten, molybdenum, chromium, and mixtures thereof. Preferably the promoter component comprises, as an element, rhenium.

The promoter component may typically be present in a quantity of at least 0.01 mmole/kg, more typically at least 0.1 mmole/kg, and preferably at least 0.5 mmole/kg, calculated as the total quantity of the element (that is rhenium, tungsten, molybdenum and/or chromium) relative to the weight of the catalyst. The promoter component may be present in a quantity of at most 50 mmole/kg, preferably at most 10 mmole/kg, more preferably at most 5 mmole/kg, calculated as the total quantity of the element relative to the weight of the catalyst. The form in which the promoter component may be deposited onto the carrier is not material to the invention. For example, the promoter component may suitably be provided as an oxide or as an oxyanion, for example, as a rhenate, perrhenate, or tungstate, in salt or acid form.

When the catalyst comprises a rhenium containing promoter component, rhenium may typically be present in a quantity of at least 0.1 mmole/kg, more typically at least 0.5 mmole/kg, and preferably at least 1.0 mmole/kg, in particular at least 1.5 mmole/kg, calculated as the quantity of the element relative to the weight of the catalyst. Rhenium is typically present in a quantity of at most 5.0 mmole/kg, preferably at most 3.0 mmole/kg, more preferably at most 2.0 mmole/kg, in particular at most 1.5 mmole/kg.

Further, when the catalyst comprises a rhenium containing promoter component, the catalyst may preferably comprise a rhenium co-promoter, as a further component deposited on the carrier. Suitably, the rhenium co-promoter may be selected from components comprising an element selected from tungsten, chromium, molybdenum, sulfur, phosphorus, boron, and mixtures thereof. Preferably, the rhenium co-promoter is selected from components comprising tungsten, chromium, molybdenum, sulfur, and mixtures thereof. It is particularly preferred that the rhenium co-promoter comprises, as an element, tungsten.

The rhenium co-promoter may typically be present in a total quantity of at least 0.01 mmole/kg, more typically at least 0.1 mmole/kg, and preferably at least 0.5 mmole/kg, calculated as the element (i.e. the total of tungsten, chromium, molybdenum, sulfur, phosphorus and/or boron), relative to the weight of the catalyst. The rhenium co-promoter may be present in a total quantity of at most 40 mmole/kg, preferably at most 10 mmole/kg, more preferably at most 5 mmole/kg, on the same basis. The form in which the rhenium co-promoter may be deposited on the carrier is not material to the invention. For example, it may suitably be provided as an oxide or as an oxyanion, for example, as a sulfate, borate or molybdate, in salt or acid form.

The catalyst preferably comprises silver, the promoter component, and a component comprising a further element, deposited on the carrier. Eligible further elements may be selected from the group of nitrogen, fluorine, alkali metals, alkaline earth metals, titanium, hafnium, zirconium, vanadium, thallium, thorium, tantalum, niobium, gallium and germanium and mixtures thereof. Preferably the alkali metals are selected from lithium, potassium, rubidium and cesium. Most preferably the alkali metal is lithium, potassium and/or cesium. Preferably the alkaline earth metals are selected from calcium and barium. Typically, the further element is present in the catalyst in a total quantity of from 0.01 to 500 mmole/kg, more typically from 0.05 to 100 mmole/kg, calculated as the element on the weight of the catalyst. The further elements may be provided in any form. For example, salts of an alkali metal or an alkaline earth metal are suitable.

As used herein, the quantity of alkali metal present in the catalyst is deemed to be the quantity insofar as it can be extracted from the catalyst with de-ionized water at 100° C. The extraction method involves extracting a 10-gram sample of the catalyst three times by heating it in 20 ml portions of de-ionized water for 5 minutes at 100° C. and determining in the combined extracts the relevant metals by using a known method, for example atomic absorption spectroscopy.

As used herein, the quantity of alkaline earth metal present in the catalyst is deemed to the quantity insofar as it can be extracted from the catalyst with 10% w nitric acid in de-ionized water at 100° C. The extraction method involves extracting a 10-gram sample of the catalyst by boiling it with a 100 ml portion of 10% w nitric acid for 30 minutes (1 atm., i.e. 101.3 kPa) and determining in the combined extracts the relevant metals by using a known method, for example atomic absorption spectroscopy. Reference is made to U.S. Pat. No. 5,801,259, which is incorporated herein by reference.

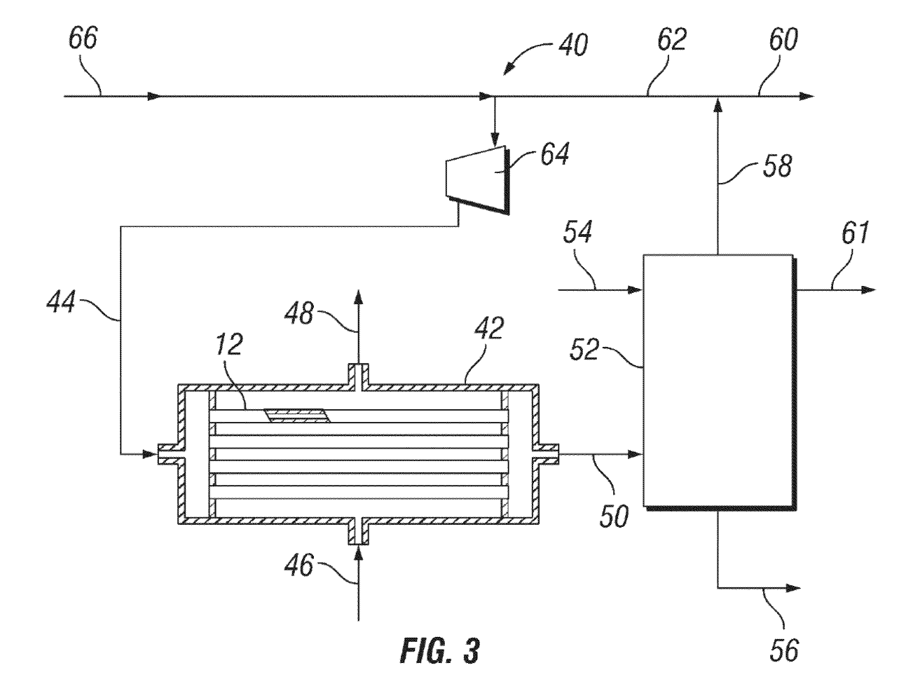

FIG. 3 is a schematic representation showing a typical ethylene oxide manufacturing system 40 with a shell-and-tube heat exchanger 42 which is equipped with various non-thermocouple reactor tubes as depicted in FIG. 1 and various thermocouple reactor tubes as depicted in FIG. 2. The various reactor tubes are grouped together into a tube bundle for insertion into the shell of a shell-and-tube heat exchanger. The skilled person will understand that the catalyst particles may be packed into the individual elongated tubes such that the elongated tubes and their contents provide the same resistivity when a gas flow passes through the elongated tubes. The number of elongated tubes present in the shell-and-tube heat exchanger 42 is typically in the range of from 1,000 to 12,000, more typically in the range of from 2,000 to 10,000. Generally, such elongated tubes are in a substantially parallel position relative to each other. Ethylene oxide manufacturing system 40 may comprise one or more shell-and-tube heat exchangers 42, for example two, three or four.

A feedstream comprising ethylene and oxygen is charged via conduit 44 to the tube side of shell-and-tube heat exchanger 42 wherein it is contacted with the catalyst bed contained therein within elongated tubes 12. The shell-and-tube heat exchanger 42 is typically operated in a manner which allows an upward or downward flow of gas through the catalyst bed. The heat of reaction is removed and control of the reaction temperature, that is the temperature within the catalyst bed, is achieved by use of a heat transfer fluid, for example oil, kerosene or water, which is charged to the shell side of shell-and-tube heat exchanger 42 by way of conduit 46 and the heat transfer fluid is removed from the shell of shell-and-tube heat exchanger 42 through conduit 48.

The reaction product comprising ethylene oxide, unreacted ethylene, unreacted oxygen and, optionally, other reaction products such as carbon dioxide and water, is withdrawn from the reactor system tubes of shell-and-tube heat exchanger 42 through conduit 50 and passes to separation system 52. Separation system 52 provides for the separation of ethylene oxide from ethylene and, if present, carbon dioxide and water. An extraction fluid such as water can be used to separate these components and is introduced to separation system 52 by way of conduit 54. The enriched extraction fluid containing ethylene oxide passes from separation system 52 through conduit 56 while unreacted ethylene and carbon dioxide, if present, passes from separation system 52 through conduit 58. Separated carbon dioxide passes from separation system 52 through conduit 61. A portion of the gas stream passing through conduit 58 can be removed as a purge stream through conduit 60. The remaining gas stream passes through conduit 62 to recycle compressor 64. A stream containing ethylene and oxygen passes through conduit 66 and is combined with the recycle ethylene that is passed through conduit 62 and the combined stream is passed to recycle compressor 64. Recycle compressor 64 discharges into conduit 44 whereby the discharge stream is charged to the inlet of the tube side of the shell-and-tube heat exchanger 42. Ethylene oxide produced may be recovered from the enriched extraction fluid, for example by distillation or extraction.

The ethylene concentration in the feedstream passing through conduit 44 may be selected within a wide range. Typically, the ethylene concentration in the feedstream will be at most 80 mole-%, relative to the total feed. Preferably, it will be in the range of from 0.5 to 70 mole-%, in particular from 1 to 60 mole-%, on the same basis. As used herein, the feedstream is considered to be the composition which is contacted with the catalyst particles.

The present epoxidation process may be air-based or oxygen-based, see “Kirk-Othmer Encyclopedia of Chemical Technology”, 3^{rd} edition, Volume 9, 1980, pp. 445-447. In the air-based process air or air enriched with oxygen is employed as the source of the oxidizing agent while in the oxygen-based processes high-purity (at least 95 mole-%) oxygen is employed as the source of the oxidizing agent. Presently most epoxidation plants are oxygen-based and this is a preferred embodiment of the present invention.

The oxygen concentration in the feedstream passing through conduit 44 may be selected within a wide range. However, in practice, oxygen is generally applied at a concentration which avoids the flammable regime. Typically, the concentration of oxygen applied will be within the range of from 1 to 15 mole-%, more typically from 2 to 12 mole-% of the total feed. The actual safe operating ranges depend, along with the feedstream composition, also on the reaction conditions such as the reaction temperature and the pressure.

An organic halide may be present in the feedstream passing through conduit 44 as a reaction modifier for increasing the selectivity, suppressing the undesirable oxidation of ethylene or ethylene oxide to carbon dioxide and water, relative to the desired formation of ethylene oxide. Fresh organic halide is suitably fed to the process through conduit 66. Organic halides are in particular organic bromides, and more in particular organic chlorides. Preferred organic halides are chlorohydrocarbons or bromohydrocarbons. More preferably they are selected from the group of methyl chloride, ethyl chloride, ethylene dichloride, ethylene dibromide, vinyl chloride or a mixture thereof. Most preferred are ethyl chloride and ethylene dichloride.

The organic halides are generally effective as reaction modifier when used in low concentration in the feed, for example up to 0.01 mole-%, relative to the total feed. It is preferred that the organic halide is present in the feedstream at a concentration of at most 50×10⁻⁴ mole-%, in particular at most 20×10⁻⁴ mole-%, more in particular at most 15×10⁻⁴ mole-%, relative to the total feed, and preferably at least 0.2×10⁻⁴ mole-%, in particular at least 0.5×10⁻⁴ mole-%, more in particular at least 1×10⁻⁴ mole-%, relative to the total feed.

In addition to ethylene, oxygen and the organic halide, the feedstream may contain one or more optional components, for example carbon dioxide, inert gases and saturated hydrocarbons. Carbon dioxide generally has an adverse effect on the catalyst activity. Advantageously, separation system 52 is operated in such a way that the quantity of carbon dioxide in the feedstream through conduit 44 is low, for example, below 2 mole-%, preferably below 1 mole-%, or in the range of from 0.2 to 1 mole-%. Inert gases, for example nitrogen or argon, may be present in the feedstream passing through conduit 44 in a concentration of from 30 to 90 mole-%, typically from 40 to 80 mole-%. Suitable saturated hydrocarbons are methane and ethane. If saturated hydrocarbons are present, they may be present in a quantity of up to 80 mole-%, relative to the total feed, in particular up to 75 mole-%. Frequently they are present in a quantity of at least 30 mole-%, more frequently at least 40 mole-%. Saturated hydrocarbons may be employed in order to increase the oxygen flammability limit. Olefins other than ethylene may be present in the feedstream, for example in a quantity of less than 10 mole-%, in particular less than 1 mole-%, relative to the quantity of ethylene. However, it is preferred that ethylene is the single olefin present in the feedstream.

The epoxidation process may be carried out using reaction temperatures selected from a wide range. Preferably the reaction temperature is in the range of from 150 to 340° C., more preferably in the range of from 180 to 325° C. Typically, the shell-side heat transfer liquid has a temperature which is typically 1 to 15° C., more typically 2 to 10° C. lower than the reaction temperature.

In order to reduce the effects of deactivation of the catalyst, the reaction temperature may be increased gradually or in a plurality of steps, for example in steps of from 0.1 to 20° C., in particular 0.2 to 10° C., more in particular 0.5 to 5° C. The total increase in the reaction temperature may be in the range of from 10 to 140° C., more typically from 20 to 100° C. The reaction temperature may be increased typically from a level in the range of from 150 to 300° C., more typically from 200 to 280° C., when a fresh catalyst is used, to a level in the range of from 230 to 340° C., more typically from 240 to 325° C., when the catalyst has decreased in activity due to ageing.

The epoxidation process is preferably carried out at a pressure in the inlet tube end 26 in the range of from 1000 to 3500 kPa. “GHSV” or Gas Hourly Space Velocity is the unit volume of gas at normal temperature and pressure (0° C., 1 atm, i.e. 101.3 kPa) passing over one unit of the total volume of catalyst bed per hour. Preferably, the GHSV is in the range of from 1500 to 10000 Nm³/(m³h). Preferably, the process is carried out at a work rate in the range of from 0.5 to 10 kmole ethylene oxide produced per m³ of the total catalyst bed per hour, in particular 0.7 to 8 kmole ethylene oxide produced per m³ of the total catalyst bed per hour, for example 5 kmole ethylene oxide produced per m³ of the total catalyst bed per hour.

The ethylene oxide produced in the epoxidation process may be converted, for example, into ethylene glycol, an ethylene glycol ether or an ethanol amine.

The conversion into ethylene glycol or the ethylene glycol ether may comprise, for example, reacting the ethylene oxide with water, suitably using an acidic or a basic catalyst. For example, for making predominantly the ethylene glycol and less ethylene glycol ether, the ethylene oxide may be reacted with a ten-fold molar excess of water, in a liquid phase reaction in presence of an acid catalyst, e.g. 0.5-1.0% w sulfuric acid, based on the total reaction mixture, at 50-70° C. at 100 kPa absolute, or in a gas phase reaction at 130-240° C. and 2000-4000 kPa absolute, preferably in the absence of a catalyst. If the proportion of water is lowered the proportion of ethylene glycol ethers in the reaction mixture is increased. The ethylene glycol ethers thus produced may be a di-ether, tri-ether, tetra-ether or a subsequent ether. Alternative ethylene glycol ethers may be prepared by converting the ethylene oxide with an alcohol, in particular a primary alcohol, such as methanol or ethanol, by replacing at least a portion of the water by the alcohol.

The ethylene oxide may be converted into ethylene glycol by first converting the ethylene oxide into ethylene carbonate by reacting it with carbon dioxide, and subsequently hydrolyzing the ethylene carbonate to form ethylene glycol. For applicable methods, reference is made to U.S. Pat. No. 6,080,897, which is incorporated herein by reference.

The conversion into the ethanol amine may comprise reacting ethylene oxide with an amine, such as ammonia, an alkyl amine or a dialkyl amine. Anhydrous or aqueous ammonia may be used. Anhydrous ammonia is typically used to favor the production of mono ethanol amine. For methods applicable in the conversion of ethylene oxide into the ethanol amine, reference may be made to, for example U.S. Pat. No. 4,845,296, which is incorporated herein by reference.

Ethylene glycol and ethylene glycol ethers may be used in a large variety of industrial applications, for example in the fields of food, beverages, tobacco, cosmetics, thermoplastic polymers, curable resin systems, detergents, heat transfer systems, etc. Ethanol amines may be used, for example, in the treating (“sweetening”) of natural gas.

Unless specified otherwise, the organic compounds mentioned herein, for example the olefins, ethylene glycol ethers, ethanol amines and organic halides, have typically at most 40 carbon atoms, more typically at most 20 carbon atoms, in particular at most 10 carbon atoms, more in particular at most 6 carbon atoms. As defined herein, ranges for numbers of carbon atoms (i.e. carbon number) include the numbers specified for the limits of the ranges.

The following examples are intended to illustrate the advantages of the present invention and are not intended to unduly limit the scope of the invention.

### Example I

In the present invention, the first inventive feature is to specify a requirement that the gas flow per unit mass of catalyst in the thermocouple reactor tubes be substantially equal to that of the non-thermocouple reactor tubes. This will insure substantially equivalent reactant conversion across both types of reactor tubes. Next one must combine measurements of pressure drop and loading density of various catalysts to determine the expected differential in gas flow or pressure drop between the thermocouple reactor tubes and the non-thermocouple reactor tubes. Then one utilizes measurements of pressure drop characteristics of different types of inert materials to predict what type and amount of inert materials need to be loaded on top of the catalyst in the thermocouple reactor tubes to achieve the equivalent gas flow per unit mass of catalyst under normal reactor operating conditions. Finally, one calculates a pressure drop value that should be achieved in the pressure drop checks that are conducted on the reactor tubes in commercial reactors after loading, but prior to startup so that the proper loading of the reactor tubes can be verified in the field before placing the reactor in service.

The following illustrates one specific example according to the present invention. Pilot plant experiments were conducted in a commercially representative reactor with a reactor tube having a 45 mm inner diameter. In the first part of the experiment, the reactor tube was loaded with 18.33 kg of fresh catalyst. The tube packing density was 1088 kg/m³ and the catalyst bed height was 10.7 m. No thermocouple was placed in the catalyst bed during this phase of experimentation. Gas flow was started to the reactor and it was heated to 230° C. where it was operated for 117 days with a feed gas composition of 7.3 mol % oxygen, 35 mol % ethylene, and 0.75 mol % carbon dioxide and an outlet pressure of 14.1 barg. The total gas flow rate to the reactor was 44.78 normal m³/hr and the ethyl chloride concentration was adjusted during the course of the first phase to achieve optimal catalyst performance. At the end of the first part of the experimentation, the outlet oxygen concentration was measured to be 4.53 mol % as shown in FIG. 4 and all conditions as described. After operation for 2810 hours, the reactor was stopped and the catalyst was unloaded and saved. After installation of an axial thermocouple (6.35 mm OD) in the center of the reactor tube, the same catalyst was reloaded. Due to the presence of the thermocouple, the tube packing density of the catalyst decreased from 1088 kg/m³ to 1042 kg/m³. The reactor was then restarted and the coolant temperature and ethyl chloride feed concentration were returned to exactly the same conditions as were present prior to installation of the thermocouple. The reactor was operated on temperature control at 230° C. with constant ethyl chloride, oxygen, ethylene, carbon dioxide feed concentrations, and outlet pressure while the gas flow rate through the catalyst bed was varied from 1.9-2.9 Nm³/hr/kg catalyst.

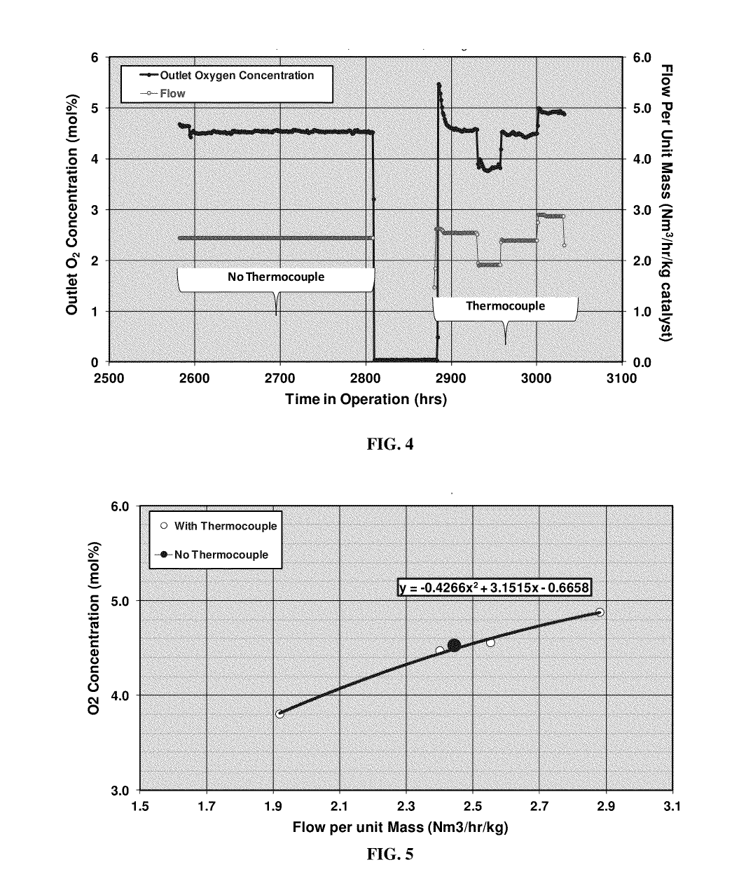

To determine the point at which both the thermocouple reactor tube the non-thermocouple reactor tube exhibited equal outlet oxygen concentration, the steady state results were plotted in FIG. 5, as outlet oxygen concentration versus flow per unit mass. A 2^{nd} order polynomial fit through the data points for the thermocouple reactor tube data was used to calculate that the outlet oxygen concentration of the thermocouple reactor tube would be 4.53% at a flow per unit mass of catalyst of 2.48 Nm³/hr/kg catalyst. This is within experimental error of the observed value of 2.44 Nm³/hr/kg catalyst that was measured for the non-thermocouple reactor tube.

After verification that the flow per unit mass of catalyst must be substantially equivalent in order to achieve equal outlet oxygen concentration from thermocouple reactor tubes and non-thermocouple reactor tubes, the next step is to determine how the resistance to flow in the thermocouple reactor tubes must be adjusted in order to achieve equal flow per unit mass of catalyst. In order to calculate the required adjustment, the following equation is used:

$$\frac{Δ P}{L} = C ρ {V}_{o}^{2} \\ Where , \\ \frac{Δ P}{L} is the pressure drop per unit reactor length \\ {V}_{o} is the superficial gas velocity , \\ ρ is the gas density , and \\ C , is the length weighted average resistivity or resistance to flow . \qquad ( 1 )$$

A common approach to adjust the resistance to flow of the thermocouple reactor tubes to give the desired gas flow rate relative to the non-thermocouple reactor tubes is to add measured amounts of inert material to the top of the thermocouple reactor tube. For a tube in which more than one material is loaded, a weighted average resistivity can be used that where the total tube resistivity is a length weighted average of each material packed in the tube. The resistivity values for several inert materials ranging in diameter from 1.6 mm to 6.4 mm were measured in a 45 mm ID tube containing a centered 6.35 mm OD thermocouple and are shown in Table 1.

TABLE 1

| | | Non Thermocouple |
|---|---|---|
| | Thermocouple Tube | Tube Resistivity |
| Material | Resistivity (bar s²/kg) | (bar s²/kg) |
| 8 mm Catalyst Pellets | 0.0104 | 0.0125 |
| 1.6 mm OD cylinders | 0.1110 | 0.1288 |
| 3.2 mm OD spheres | 0.0412 | 0.0629 |
| 6.4 mm OD spheres | 0.0120 | 0.0295 |
| 9.5 mm OD spheres | Not measured | 0.0213 |
| 12.7 mm OD spheres | Not measured | 0.0185 |

A 45 mm ID non-thermocouple reactor tube loaded with 10.7 m of catalyst pellets and then 1.1 m of inert material in the form of 12.7 mm spheres on top of the catalyst pellets will be used as an example. Using Equation 1 and process conditions of 45.2 Nm³/hr flow through the tube at 235° C. and inlet pressure of 16.3 barg, the pressure drop was calculated to be 1.93 bar and the flow per unit mass of catalyst with a tube packing density of the 8 mm catalyst of 1088 kg/m³ is 2.44 Nm³/hr/kg. During normal operation, the thermocouple reactor tubes will have the same inlet and outlet pressure as the non-thermocouple reactor tubes, thus the resistance to flow must be adjusted so the actual flow through the thermocouple reactor tube meets the desired target. The presence of the thermocouple in the thermocouple reactor tube also reduces the packing density of the catalyst. In this example the density was 1042 kg/m³. Using a fixed pressure drop of 1.93 bar, it was calculated that 0.15 m of 6.4 mm OD spheres, 0.067 m of 3.2 mm OD spheres, and 0.823 m of 1.6 mm OD cylinders would be required to achieve 2.44 Nm³/hr/kg 2, or essentially equal gas flow per unit mass of catalyst. The weighted average resistivity value for the non-thermocouple reactor tube was calculated to be 0.01783 bar s²/kg while for the thermocouple reactor tube it was 0.02211 bar s²/kg based on this loading information and Table 1 resistivity values.

For a constant flow pressure drop check conducted at 28.3 Nm³/hr and 1.013 barg outlet pressure on the tube, the pressure drop of the non-thermocouple reactor tubes is calculated to be 1.23 bar using equation 1. For the thermocouple reactor tube, using 0.02211 bar s²/kg resistivity and Equation 1 gives a calculated pressure drop in the check of 1.52 bar. This is 124% of the non-thermocouple reactor tube pressure drop. Thus, in the post reactor loading checks the thermocouple reactor tube target pressure drop would have to be 124% of the non-thermocouple reactor tubes to achieve a constant flow per unit mass during normal reactor operation.

1. A process for improving the control of a fixed bed, multi-tubular reactor for the preparation of ethylene oxide wherein a gaseous stream comprising ethylene and an oxygen-containing gas is passed through a multi-tubular reactor which comprises (a) thermocouple reactor tubes containing catalyst and an inert material loaded on top of the catalyst and (b) non-thermocouple reactor tubes containing catalyst, wherein prior to start-up and loading of the reactor tubes:
a. the gas flow per unit mass of catalyst in the thermocouple reactor tubes is specified as being substantially equal to the gas flow per unit mass of catalyst in the non-thermocouple reactor tubes;
b. the expected pressure drop and loading density of the catalyst is calculated to determine the expected differential in gas flow and/or pressure drop between the thermocouple reactor tubes and the non-thermocouple reactor tubes;
c. the pressure drop characteristics of the inert material is established to determine the amount of inert material to be loaded on top of the catalyst in the thermocouple reactor tubes and in the non-thermocouple reactor tubes to achieve the equivalent gas flow per unit catalyst in the non-thermocouple reactor tubes and in the thermocouple reactor tubes under normal operating conditions; and
d. a pressure drop value across the thermocouple reactor tubes that should be achieved in pressure drop checks that are to be conducted on the reactor tubes after loading is calculated.

2. The process of claim 1 wherein the reactor tubes are then loaded with the catalyst and inerts as determined by the prior calculations, and the pressure drop across thermocouple-containing tubes and non-thermocouple-containing tubes are measured to determine any difference in pressure drop across the two types of tubes.

3. The process of claim 2 wherein the amount of inerts in the thermocouple-containing tubes is adjusted to achieve substantially equivalent flow per unit mass as found in the non-thermocouple-containing tubes.

4. The process of claim 3 wherein the fixed-bed multi-tubular reactor is equipped with 5 to 50 tubes containing a thermocouple out of a total number of tubes in the reactor comprising 1,000 to 12,000 reactor tubes.

5. The process of claim 4 wherein said catalyst comprises a carrier and, deposited on the carrier, silver, a rhenium promoter, a first co-promoter, and a second co-promoter, wherein:
a. the quantity of the rhenium promoter deposited on the carrier is greater than 1 mmole/kg, relative to the weight of the catalyst;
b. the first co-promoter is selected from sulfur, phosphorus, boron, and mixtures thereof; and
c. the second co-promoter is selected from tungsten, molybdenum, chromium, and mixtures thereof.

6. The process of claim 5 wherein the total quantity of the first co-promoter and the second co-promoter deposited on the carrier is at most 10.0 mmole/kg, relative to the weight of the catalyst; and said carrier has a monomodal, bimodal or multimodal pore size distribution, with a pore diameter range of 0.01-200 μm, a specific surface area of 0.03-10 m²/g, a pore volume of 0.2-0.7 cm³/g, wherein the median pore diameter of said carrier is 0.1-100 μm and has a water absorption of 10-80%.

7. The process of claim 6 wherein the inerts are selected from the group consisting of spheres or cylinders with dimensions in the range of 1 mm to 7 mm.

8. The process of claim 7 wherein the tube inner diameter is from 30 to 50 mm and the axial thermocouple diameter is from 3 to 10 mm.

9. (canceled)

10. (canceled)
